(19) United States
(12) Patent Application Publication
(10) Pub. No.: US 2013/0030045 A1
(43) Pub. Date: Jan. 31, 2013

(54) METHOD OF PREVENTING AND TREATING OSTEOPOROSIS

(71) Applicants: Yu-Hui TSAI, Taipei City (TW); Yu-Wei Wu, Taipei City (TW); Yu-Hsiang Lin, Taipei City (TW); Wen-Fu Lai, Taipei City (TW); Shih-Ching Chen, Taipei City (TW)

(72) Inventors: Yu-Hui TSAI, Taipei City (TW); Yu-Wei Wu, Taipei City (TW); Yu-Hsiang Lin, Taipei City (TW); Wen-Fu Lai, Taipei City (TW); Shih-Ching Chen, Taipei City (TW)

(21) Appl. No.: 13/567,934

(22) Filed: Aug. 6, 2012

Related U.S. Application Data
(63) Continuation of application No. 13/194,379, filed on Jul. 29, 2011.

(51) Int. Cl. A61K 31/353; A61P 19/10

(52) U.S. Cl. 514/456

(57) ABSTRACT

The invention discloses a method of preventing and treating osteoporosis. The method comprises a step of enhancing osteoblast differentiation and inhibiting osteoclast differentiation by administering a pharmaceutically effective amount of a composition comprising 6-hydroxy flavone compound or 7-methoxy flavone compound, or at least one pharmaceutically acceptable salt thereof to a subject.

### CROSS-REFERENCE TO RELATED APPLICATION

This application is a continuation of prior application Ser. No. 13/194,379, filed on Jul. 29, 2011. The patent application identified above is incorporated here by reference in its entirety to provide continuity of disclosure.

### BACKGROUND OF THE INVENTION

1. Field of the Invention

The present invention relates to a field of bone regeneration, and in particular to a method of preventing and treating osteoporosis.

2. Description of Related Art

Bone provides support, protection, mobility and hematopoiesis functions in the body. Bone tissue is dynamic: healthy bones require continuous formation and resorption to regulate mineral homeostasis. That is, bone in the body is unceasingly decomposed and regenerated, and this bone metabolism process is known as bone remodeling. Bone is mainly composed of bone cells and bone matrix, and the bone cells comprise osteoblasts, osteoclasts and osteocytes. The osteoblasts are derived from the differentiation of bone marrow mesenchymal stem cells and can secrete the bone matrix to be mineralized so as to form new bone; the osteoclasts are derived from the differentiation of monocytes, and can perform bone resorption and decomposition and release minerals in the bone to blood; both osteoblasts and osteoclasts are useful for bone retention in the body.

However, an imbalance in bone formation and bone resorption may lead to osteoporosis. Osteoporosis is a kind of metabolic bone disease characterized by reduced bone mass density. Aging and postmenopause are among the most importance risk factors for developing osteoporosis. In general, the bone mass density of human will gradually increase from birth, and during puberty, the bone mass density rises faster due to the effect of sexual hormones. To about 35 years of age, the bone mass density reaches a peak. After about 45 years old, the bone decomposition rate will be larger than the bone formation rate, resulting in gradual loss of bone mass and decreased bone mass density. When T value of the bone density falls to below −2.5, it is called as osteoporosis. Osteoporosis will make bone hollow and fragile and thus cause increased risk of fractures.

Osteoporosis is roughly divided into two kinds: primary osteoporosis and secondary osteoporosis. Primary Osteoporosis can be further divided into two types: postmenopausal (Type I) osteoporosis and senile (Type II) osteoporosis. Type I osteoporosis occurs in postmenopausal women; Type II osteoporosis generally occurs in the elderly after the age of 70. Secondary Osteoporosis: it could be found in any age of the male or female and is caused by factors, such as hyperthyroidism, diabetes, genetic diseases, rheumatoid arthritis, and so on. 80 percents of osteoporosis belongs to the primary osteoporosis, and particularly to postmenopausal osteoporosis.

Factors causing osteoporosis may includes: (1) Aging: bone aging with ages and thus bone mass density dropping off; (2) Sex: the bone mass density of the female being less than that of the male; (3) Underweight: the bone mass density being relatively low for people with lighter weight; (4) Decreased estrogen: estrogen stimulating bone regeneration and inhibiting bone decomposition; (5) Diets: calcium, vitamin D, or protein deficiency; excessive smoking or drinking; drinking coffee or tea; intaking high-protein or high-salt foods; (6) Irregular living habits: lack of exercise, less sun, prolonged bed rest; (7) Genetic causes: several members in a family having osteoporosis; and (8) Specific diseases: hyperthyroidism, gonadal insufficiency, rheumatoid arthritis, diabetes, liver disease patients being predisposed to get osteoporosis. The previous studies refer that once women stop menstruation, estrogen secretion decrease, thereby accelerating bone decomposition and suddenly reducing the bone mass density and resulting in osteoporosis.

Hormone replacement therapy (HRT) can increase the bone mass density of menopausal and postmenopausal women. However, HRT using estrogen also reported to make patients have nausea, and even increase the risk of breast and endometrial cancers. Alternatively, some flavonoids are known to have phytoestrogenic effects: increasing the bone mass density and reducing bone loss. Flavonoids comprise flavones, flavonols, flavanones, catechins, anthocyanidins and isoflavones. Based on the literature, flavones have functions, such as anticancer, antioxidant, anti-inflammation, prevention of heart disease. Nevertheless, few reports have addressed on the effects of flavones on osteoblast and osteoclast formation.

### SUMMARY OF THE INVENTION

An object of the present invention is to provide a compound capable of enhancing osteoblast differentiation and inhibiting osteoclast differentiation so as to achieve the effect of more efficient treatment for osteoporosis.

According to the object of the present invention, a compound for enhancing osteoblast differentiation and inhibiting osteoclast differentiation is provided. The compound comprises methoxy flavone or hydroxy flavone.

In an embodiment, methoxy flavone is 6-methoxy flavone or 7-methoxy flavone, the concentration of which is 3-60 μM, preferably 5-50 μM.

In another embodiment, hydroxy flavone comprises 6-hydroxy flavone or 7-hydroxy flavone. The concentration of 6-hydroxy flavone is 3-60 μM, preferably 5-50 μM, and more preferably 10-20 μM. The concentration of 7-hydroxy flavone is 10-20 preferably 20 μM.

Wherein, osteoblast may be preosteoblast cells or osteosarcoma cells. Osteoclasts may be preosteoclast cells.

Further, the present invention provides a pharmaceutical composition for enhancing osteoblast differentiation maturation and inhibiting osteoclast differentiation, comprising the compound as above mentioned or a pharmaceutically acceptable salt thereof, and one or more pharmaceutically acceptable carriers, additives, diluents, stabilizers or excipients.

The present invention may have one or more advantages as follows:

(1) After the compound for enhancing osteoblast differentiation maturation and inhibiting osteoclast differentiation or the pharmaceutical composition thereof according to the present invention is used, the osteogenic differentiation can be stimulated and the extent of bone deposition can be greatly increased, and the osteoclastgenic differentiation can be inhibited.

(2) The compound of the present invention not only can accelerate calcium deposition, but also quickly increase the amount of the bone deposition so as to achieve fast bone regeneration. Therefore, for adults older than 45 years old, the compound of the present invention can reduce the risk of osteoporosis, and even stimulate the effect of bone remodeling for osteoporosis patients.

(3) For the sake of facilitating bone regeneration or bone repair, methoxy flavones or hydroxy flavones in accordance with the present invention may be used with bone materials to achieve the desired effect.

### BRIEF DESCRIPTION OF THE DRAWINGS

The exemplary embodiments of the present invention will be understood more fully from the detailed description given below and from the accompanying drawings of various embodiments of the invention, which, however, should not be taken to limit the invention to the specific embodiments, but are for explanation and understanding only.

FIG. 1(A) illustrates images of cell morphology of MBMMφ cells cultured with M-CSF but without adding RANKL to induce differentiation; FIG. 1(B) illustrates cell morphology of MBMMφ cells cultured with M-CSF and RANKL using acid phosphatase stain;

FIGS. 2(A)-2(E) illustrate the chemical structures of flavone, 6-methoxy flavone, 7-methoxy flavone, 6-hydroxy flavone and 7-hydroxy flavone, respectively;

FIGS. 3(A)-3(E) illustrate s-TRAP activities in MBMMφ cells treated with different concentrations of flavone, 6-methoxy flavone, 7-methoxy flavone, 6-hydroxy flavone and 7-hydroxy flavone, respectively, in comparison with control;

FIG. 4 illustrates the cytotoxicity effects of different concentrations of 7-methoxy flavone on MBMMφ cells;

FIGS. 5(A) and 5(B) illustrate the effects of 6-methoxy flavone and 7-methoxy flavone on ALP activities of MC3T3-E1 cells at different concentrations, respectively, in comparison with control in accordance with an embodiment;

FIGS. 6(A)-6(D) illustrate the effects of flavone, 7-methoxy flavone, 6-hydroxy flavone, and 7-hydroxy flavone, respectively, on ALP activities of MC3T3-E1 cells at different concentrations in comparison with control in accordance with another embodiment;

FIGS. 7(A)-7(C) illustrate ALP activities in MC3T3-E1 cells treated with different concentrations of flavone, 7-methoxy flavone and 6-hydroxy flavone, respectively, in comparison with control in accordance with still another embodiment;

FIGS. 8(A)-8(E) illustrate the cytotoxicity effects of different concentrations of flavone, 6-methoxy flavone, 7-methoxy flavone, 6-hydroxy flavone and 7-hydroxy flavone on MC3T3-E1 cells, respectively;

FIGS. 9(A) and 9(B) respectively illustrate the images and effects of different concentrations of 7-hydroxy flavone on calcium deposition of MC3T3-E1 cells; and

FIGS. 10(A)-10(D) respectively illustrate the effects of 6-methoxy flavone, 7-methoxyl flavone, 7-hydroxy flavone, and 6-hydroxy flavone on the osteogenesis of human mesenchymal stem cells, based on calcein fluorescent test for calcium precipitation.

### DETAILED DESCRIPTION OF THE PREFERRED EMBODIMENTS

The present invention provides a compound for enhancing osteoblast differentiation and inhibiting osteoclast differentiation, and a pharmaceutical composition thereof. The compound comprises methoxy flavone or hydroxy flavone; the pharmaceutical composition comprises the compound as above mentioned or a pharmaceutically acceptable salt thereof, and one or more pharmaceutically acceptable carriers, additives, diluents, stabilizers or excipients. Wherein, osteoblast may be preosteoblast cells or osteosarcoma cells. Osteoclasts may be preosteoclast cells.

### EXAMPLES

The present invention will be better understood by reference to the following examples, which are provided as exemplary embodiments of the invention, and not by way of limitation.

Qualitative Detection of Preosteoclast Cells

In some embodiments of the present invention, preosteoclast cells used are obtained from bone marrow in hind leg bone of C57BL/6J Narl mice, called as MBMMφ cells (mouse bone marrow macrophages), and are cultured in alpha-MEM medium (including 10% FBS and 1× antibiotic-antimycotic solution) and then added with M-CSF (macrophage colony-stimulating factor) and RANKL (receptor for activation of nuclear factor kappa B ligand) for inducing cell differentiation and maturation. In view of the mature osteoclasts being polykaryocytes and significantly expressing the activity of tatrate-resistant acid phosphatase (TRAP), cells derived from MBMMφ cells in the embodiments are observed whether or not they are polykaryocytes by a microscope and are tested whether or not they have the physiological activity of osteoclaststs through acid phosphatase stain for determining whether MBMMφ cells have differentiated into mature osteoclasts.

The acid phosphatase stain comprises the following steps. The cultured medium for cells derived from MBMMφ differentiation (using 3,000 MBMMφ cells/well) is removed, and cells are washed once with saline solution and further added with 50 μl (96 well plate) 4% formaldehyde to fix for 15 minutes at room temperature. After removing formaldehyde, cells are washed twice with saline solution and further added with 50 μl TRAP dye (0.1 M acetate buffer, 30 mM sodium tartrate, 0.1% Triton X-100, 0.1 mg/ml NP and 0.3 mg/ml FR) to set for 10 minutes at 37° C. Removing TRAP dye, cells are washed twice and added with Q water, and then are observed with the microscope.

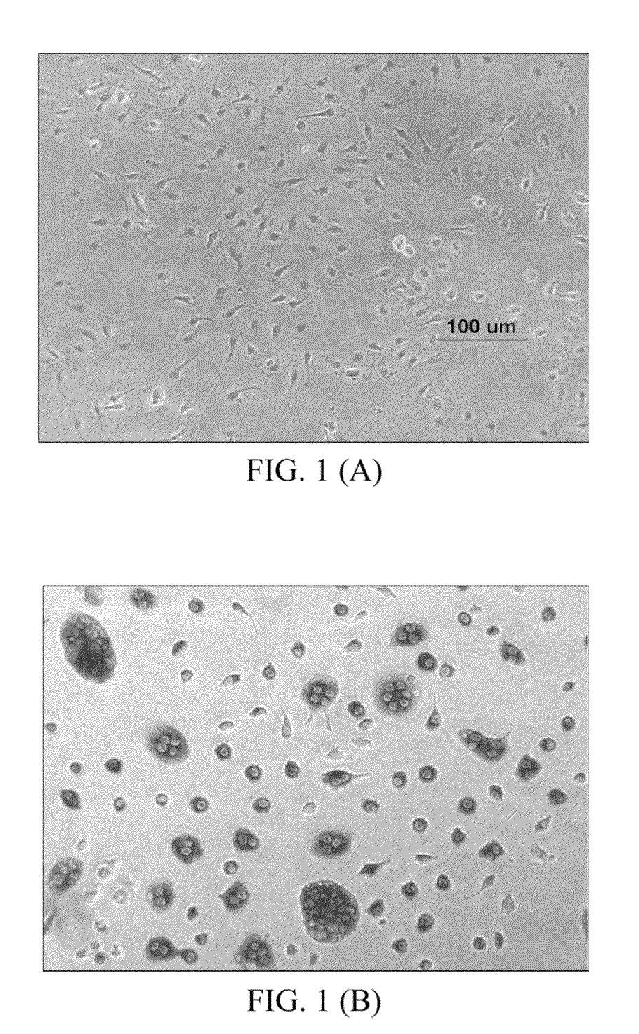

Please refer to FIG. 1(A) in which MBMMφ cells in M-CSF do not added with RANKL to induce differentiation, it can be seen that cell morphology is spindle-shape and cells are monocytes. Whereas, FIG. 1(B) in which MBMMφ cells are cultured with M-CSF and RANKL shows that cell morphology has larger cell volume and cells are polykaryocytes. After the acid phosphatase stain, FIG. 1(B) illustrates cells obviously express TRAP activity and reddish polykaryocytes refer to mature osteoclasts. Therefore, MBMMφ cells can be induced into mature osteoclasts with the medium containing M-CSF and RANKL.

TRAP Activity Assay

The cultured medium for cells derived from MBMMφ differentiation is removed, and cells are added with new medium. After incubation for 1 hour, and the activity of secretory type acid phosphatase in the medium is measured. First, 30 μl medium to be measured is taken out and added with 30 ml TRAP assay reagent (0.1 M acetate buffer, 60 mM sodium tartrate and 5 mg/ml pNPP) followed by incubating for 30 minutes at 37° C., then adding 60 μl 0.5N NaOH solution to terminate the reaction and OD value at 405 nm of each specimen being measured.

Effects of Methoxy Flavones and Hydroxyl Flavones of the Present Invention on Differentiation of MBMMφ Cells

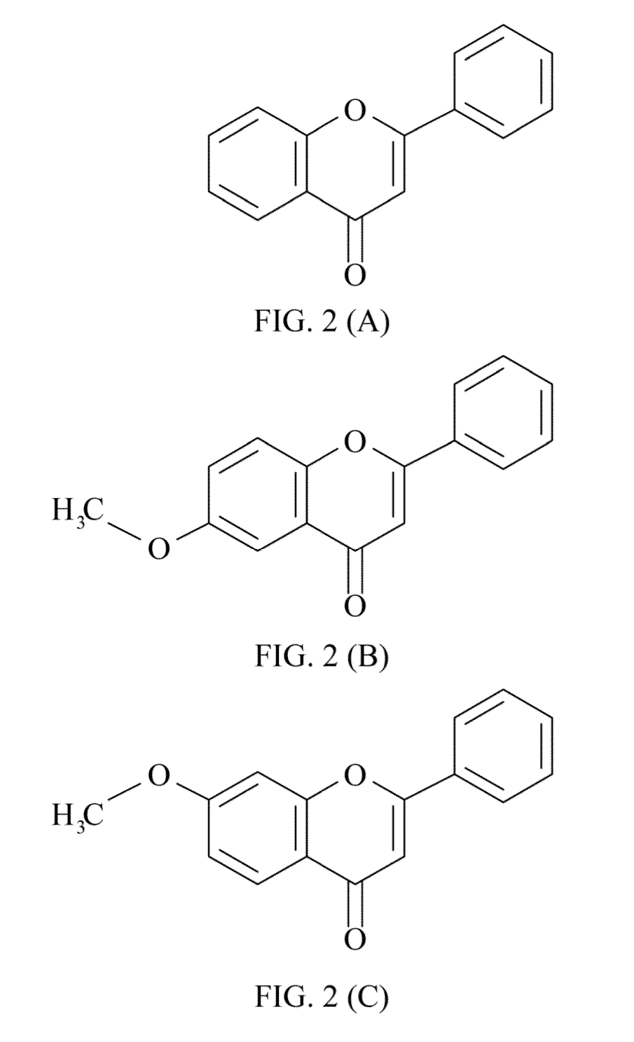

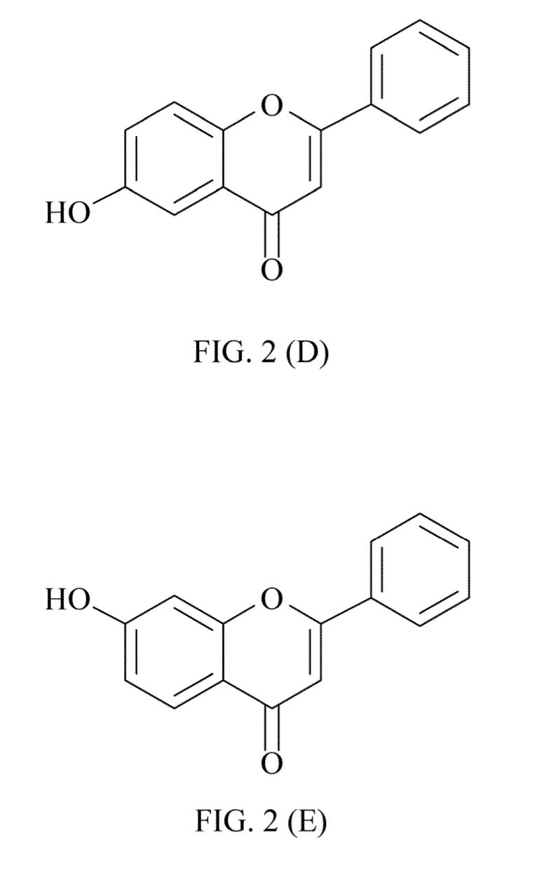

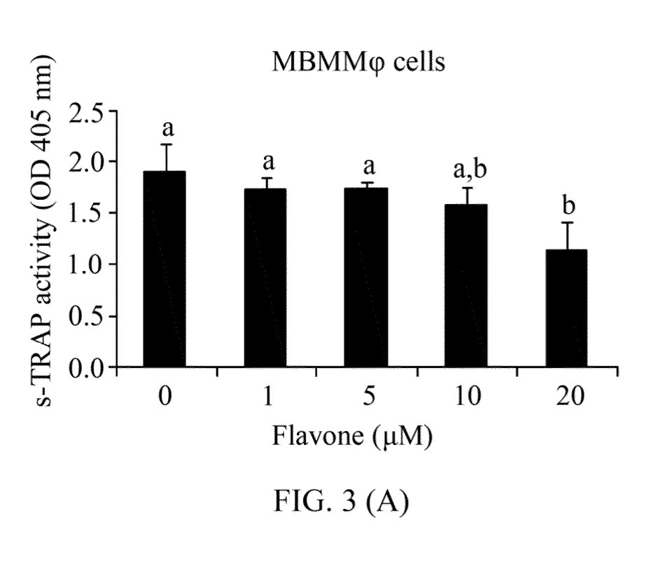

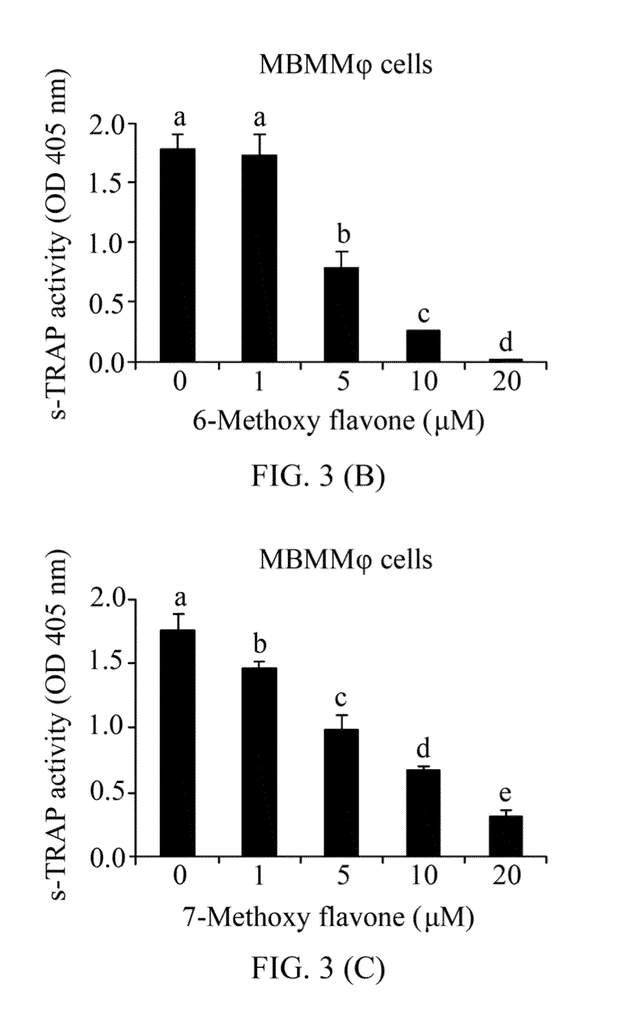

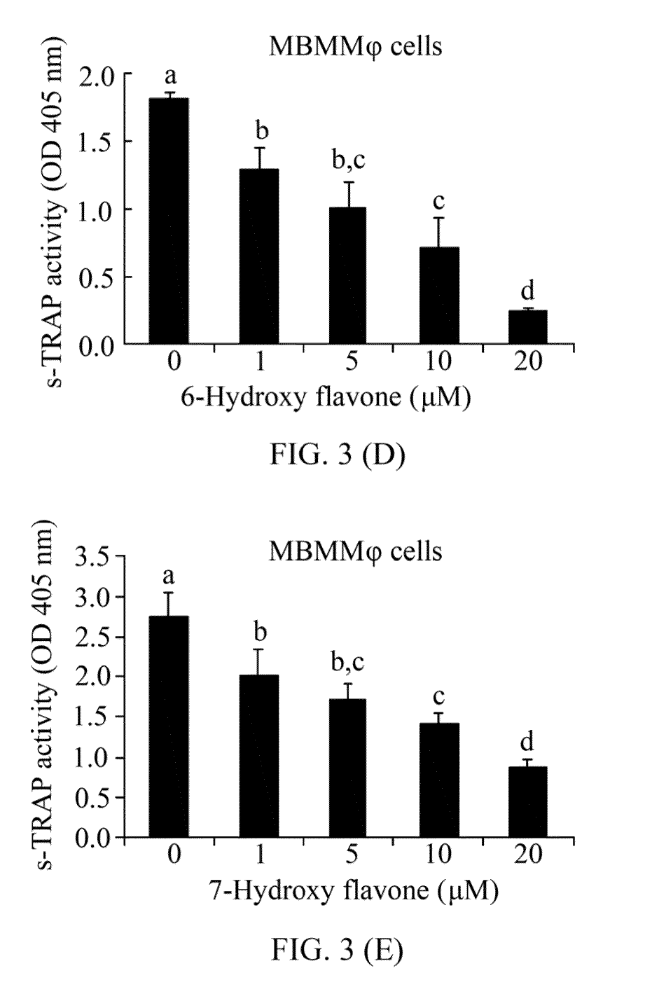

In an embodiment, different concentrations of flavone (chemical structure as shown in FIG. 2(A)), 6-methoxy flavone (FIG. 2(B)), 7-methoxy flavone (FIG. 2(C)), 6-hydroxy flavone (FIG. 2(D)) and 7-hydroxy flavone (FIG. 2(E)) are respectively added into the cultured medium of MBMMφ cells, which had been cultured in 10 ng/ml M-SCF without treating with flavone components for 5 days, for 2 days. Next, after adding 20 ng/ml RANKL and 10 ng/ml M-CSF to induce cell differentiation for another 5 days, TRAP activity assay is performed. FIGS. 3(A)-3(E) illustrate soluble-TRAP (s-TRAP) activities after cells were treated with four different concentrations (1 μM, 5 μM, 10 μM, 20 μM) of flavone, 6-methoxy flavone, 7-methoxy flavone, 6-hydroxy flavone and 7-hydroxy flavone, respectively, in comparison with control (without flavone, 6-methoxy flavone, 7-methoxy flavone, 6-hydroxy flavone, or 7-hydroxy flavone but with equal volume of vehicle). According to the results as shown in these figures, flavone does not have obvious effect (IC50>20 μM), but both 6-methoxy flavone (IC50=5.4±0.2 μM) and 7-methoxy flavone (IC50=7.1±0.6 μM) can manifestly inhibit the differentiation of MBMMφ cells at the concentration of equal to or larger than 5 μM, and both 6-hydroxy flavone (IC50=7.3±0.2 μM) and 7-hydroxy flavone (IC50=10.9±1.3 μM) also significantly inhibit the differentiation of MBMMφ cells at the concentration of equal to or larger than 10 μM.

Effects of Methoxy Flavones and Hydroxyl Flavones of the Present Invention on MBMMφ Cell Growth

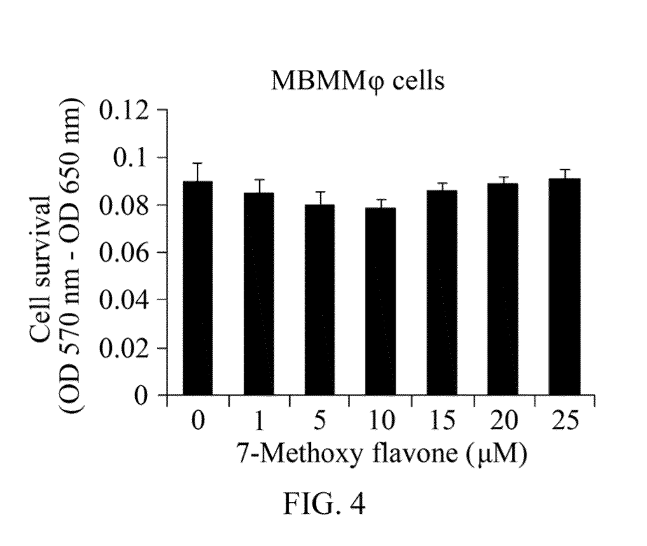

In an embodiment, different concentrations (0 μM, 1 μM, 5 μM, 10 μM, 15 μM, 20 μM, and 25 μM) of 7-methoxy flavone are respectively added into 3,000 MBMMφ cells/well in the cultured medium containing M-CSF for 1 day incubation. Next, MTT reagent is used to detect the OD value of cells so as to determine the growth situation of MBMMφ cells. The result shows 7-methoxy flavone does not affect the growth of MBMMφ cells, as shown in FIG. 4. That is, 7-methoxy flavone has no toxicity to MBMMφ cells. Moreover, flavone, 6-methoxy flavone, 6-hydroxy flavone and 7-hydroxy flavone also do not influence the growth of MBMMφ cells (not shown).

Effects of Methoxy Flavones of the Present Invention on Differentiation of RAW 264.7 Cells

In another embodiment, different concentrations of 7-methoxy flavone are respectively added into the cultured medium of RAW 264.7 cells (mouse leukaemic monocyte macrophages, another kind of preosteoclast cells) for 2 days. Next, after adding 20 ng/ml

RANKL and 10 ng/ml M-CSF to induce RAW 264.7 cell differentiation for 3 days, TRAP activity assay is then performed. Compared to control (without 7-methoxy flavone but with equal volume of vehicle), 7-methoxy flavone can significantly inhibit the differentiation of RAW 264.7 cells at the concentration of 8 μM, 16 μM, and 32 μM. Furthermore, 7-methoxy flavone has also no toxicity to RAW 264.7 cells (data not shown).

Alkaline Phosphatase (ALP) Assay

In experiments, 10,000 cells/well are cultured with alpha-MEM medium (including 10% FBS and 1× antibiotic-antimycotic solution) for 4 days at 37° C. in 5% CO₂ incubator. After adding different concentrations of methoxy flavones or hydroxy flavones to react for 4 days, the cultured medium is removed and 100 μl 4% formaldehyde is added to fix cells for 10 minutes. After washing cells, 30 μl p-nitrophenyl phosphate (pNPP) solution is added to react for 30 minutes at 37° C., and then 30 μl 0.5 N NaOH is added to stop the reaction. Finally, OD value is measured at 405 mu using ELISA reader.

Effects of Methoxy Flavones and Hydroxyl Flavones of the Present Invention on Differentiation of MC3T3-E1 Cells

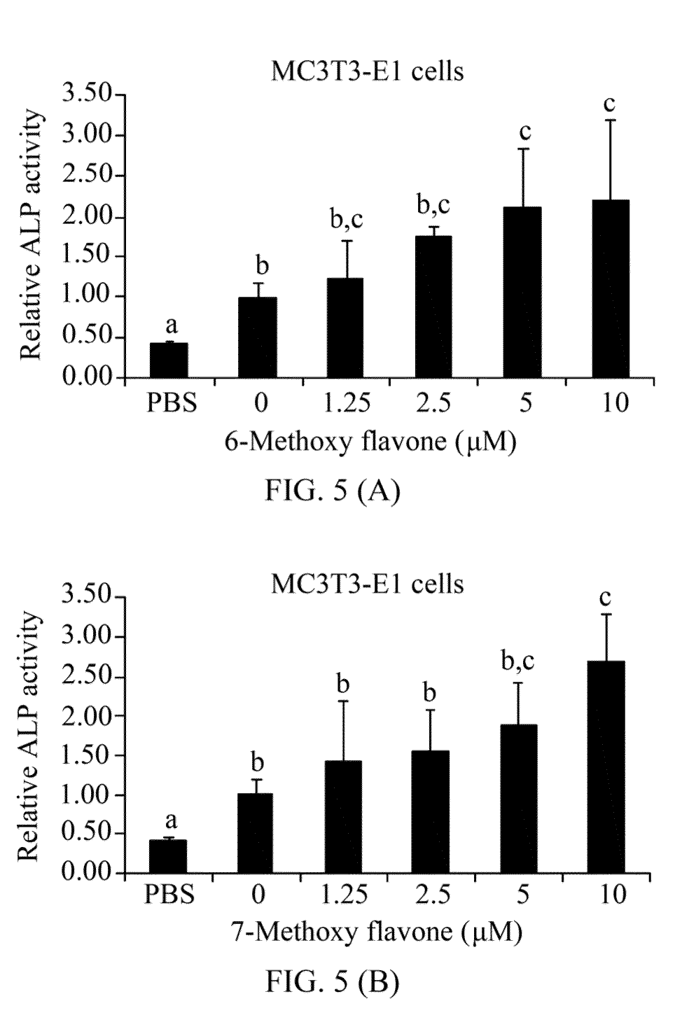

In an embodiment, different concentrations of 6-methoxy flavone and 7-methoxy flavone are respectively added into MC3T3-E1 cells (a kind of preosteoblast cells) in alpha-MEM medium (containing 10% FBS, 1× antibiotic-antimycotic solution, 10 mM β-glycerophsphate, 50 μg/ml VitC, 10⁻⁷ M dexamethasone) for 4 days to induce cell osteogenic differentiation and maturation. Because differentiated MC3T3-E1 cells will express the ALP activity, ALP activity assay is used to know the extent of cell differentiation. FIGS. 5(A) and 5(B) illustrate ALP activities at four different concentrations (1.25 μM, 2.5 μM, 5 μM, 10 μM) of 6-methoxy flavone and 7-methoxy flavone, respectively, in comparison with controls (with only PBS, or without 6-methoxy flavone or 7-methoxy flavone but with equal volume of vehicle, DMSO). As shown in the figures, both 6-methoxy flavone and 7-methoxy flavone can efficiently enhance the differentiation of MC3T3-E1 cells at the concentration of 5 μM and 10 μM.

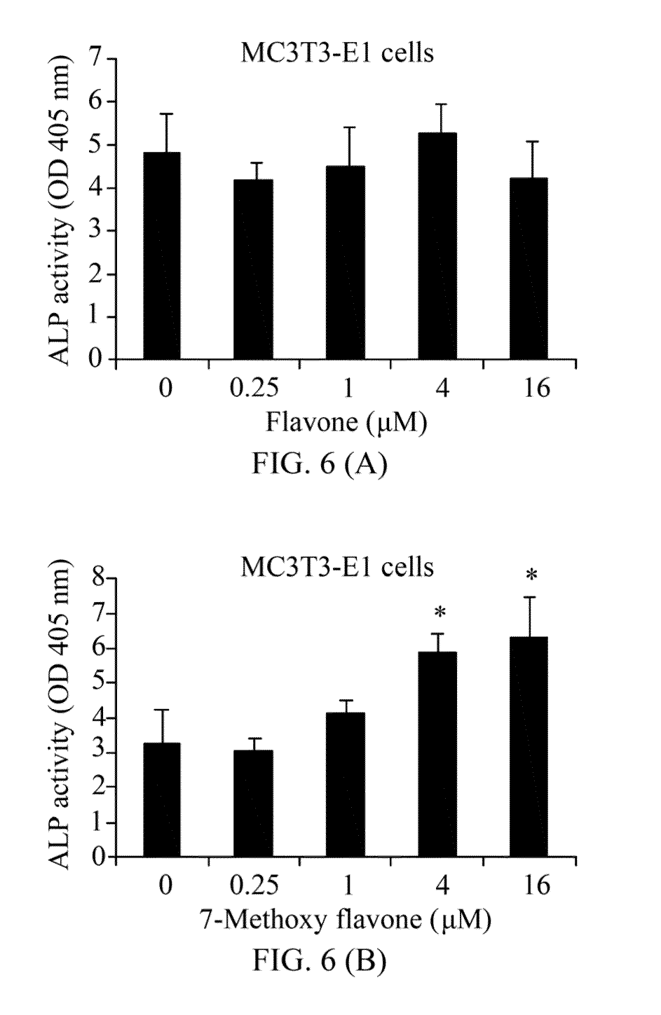

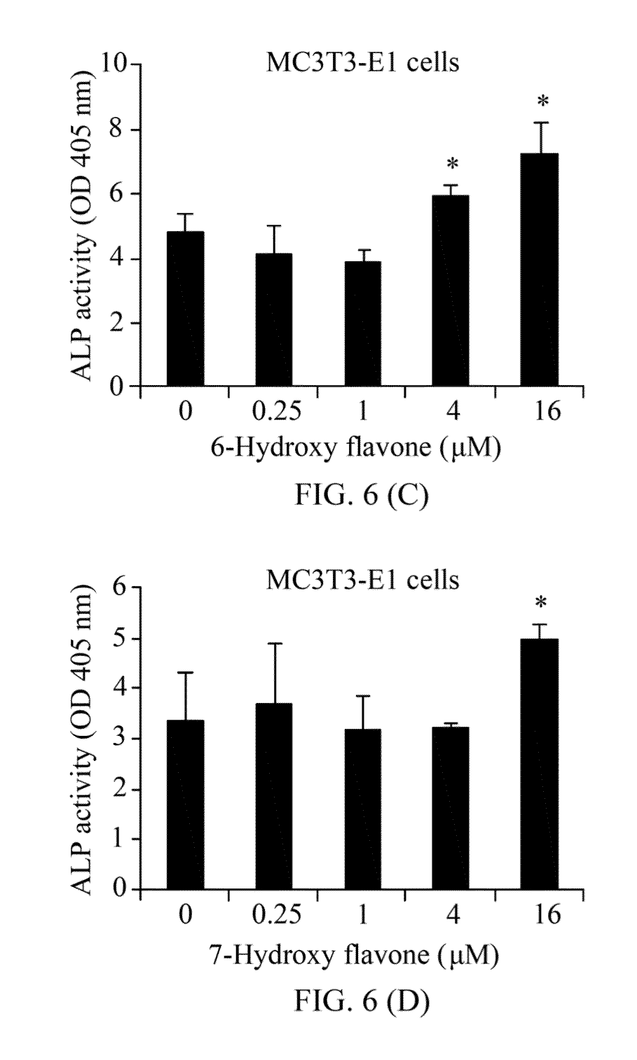

In another embodiment, different concentrations of flavone, 7-methoxy flavone, 6-hydroxy flavone, and 7-hydroxy flavone are respectively added into MC3T3-E1 cells in alpha-MEM medium with 10% FBS, 1× antibiotic-antimycotic solution, 10 mM β-glycerophsphate, 50 μg/ml Vitamin C, and 10⁻⁷ M dexamethasone for 4 days to induce cell differentiation and maturation. FIGS. 6(A)-6(D) illustrate ALP activities of MC3T3-E1 cells after exposed to four different concentrations (0.25 μM, 1 μM, 4 μM, 16 μM) of flavone, 7-methoxy flavone, 6-hydroxy flavone, and 7-hydroxy flavone, respectively, in comparison with control (without flavone, 7-methoxy flavone, 6-hydroxy flavone or 7-hydroxy flavone but with equal volume of vehicle). As shown in these figures, flavone does not stimulate the differentiation of MC3T3-E1 cells, but both 7-methoxy flavone and 6-hydroxy flavone can significantly stimulate the differentiation of MC3T3-E1 cells at the concentration of 4 μM and 16 μM, and 7-hydroxy flavone can enhance effectively the cell differentiation at 16 μM.

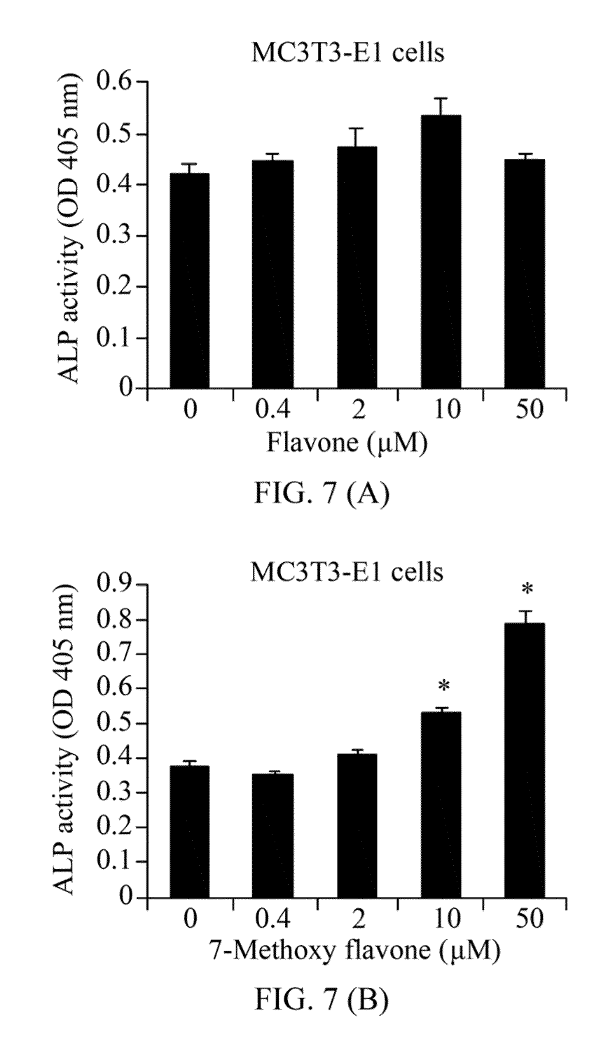

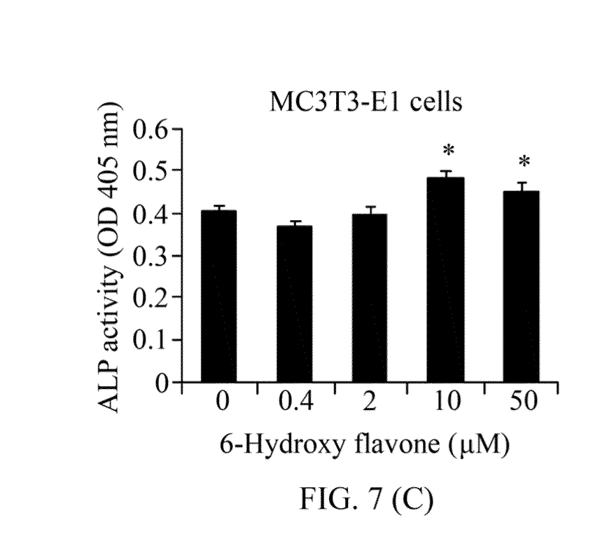

In still another embodiment, different concentrations (0.4 μM, 2 μM, 10 μM, 50 μM) of flavone, 7-methoxy flavone and 6-hydroxy flavone are respectively added into MC3T3-E1 cells in alpha-MEM medium with 10% FBS, 1× antibiotic-antimycotic solution, 10 mM β-glycerophsphate, 50 μg/ml Vitamin C, and 10⁻⁷ M dexamethasone for 4 day incubation to induce cell differentiation and maturation. FIGS. 7(A)-7(C) illustrate ALP activities of MC3T3-E1 cells after exposed to four different concentrations of flavone, 7-methoxy flavone and 6-hydroxy flavone, respectively, in comparison with control (without flavone, 7-methoxy flavone or 6-hydroxy flavone, but with vehicle DMSO). The results show flavone does not significantly stimulate the differentiation of MC3T3-E1 cells, but both 7-methoxy flavone and 6-hydroxy flavone obviously stimulate the differentiation of MC3T3-E1 cells at the concentration of 10 μM and 50 μM; especially the 7-methoxyflavone.

In sum of the above results, it can be known that methoxy flavone and hydroxyl flavone in accordance with the present invention certainly enhance the differentiation of preosteoblast cells.

Effects of Methoxy Flavones and Hydroxyl Flavones of the Present Invention on MC3T3-E1 Cell Growth

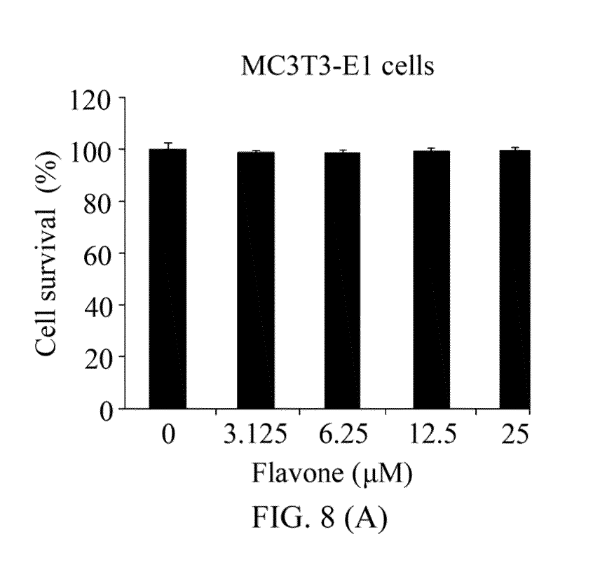

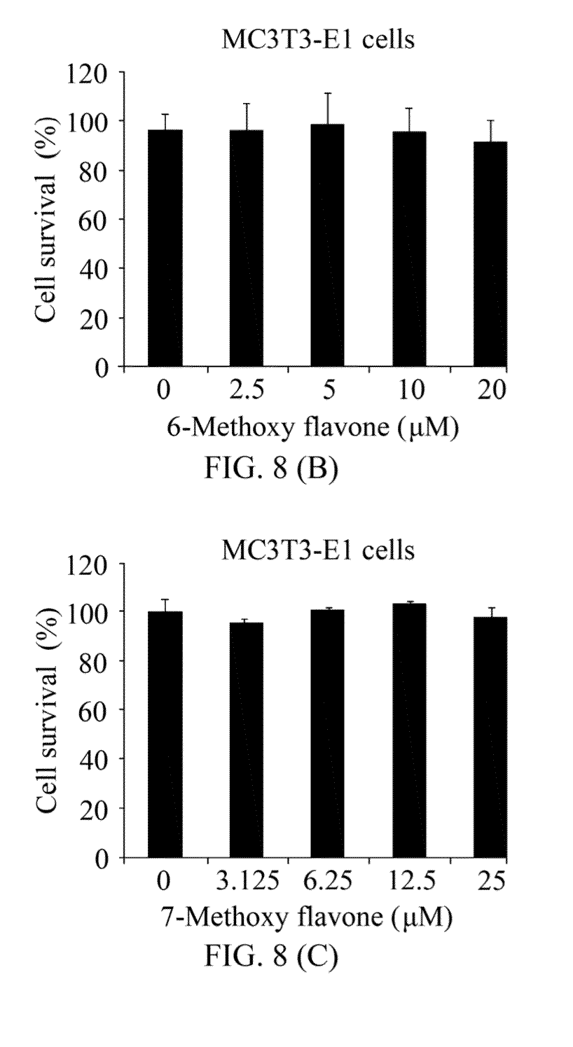

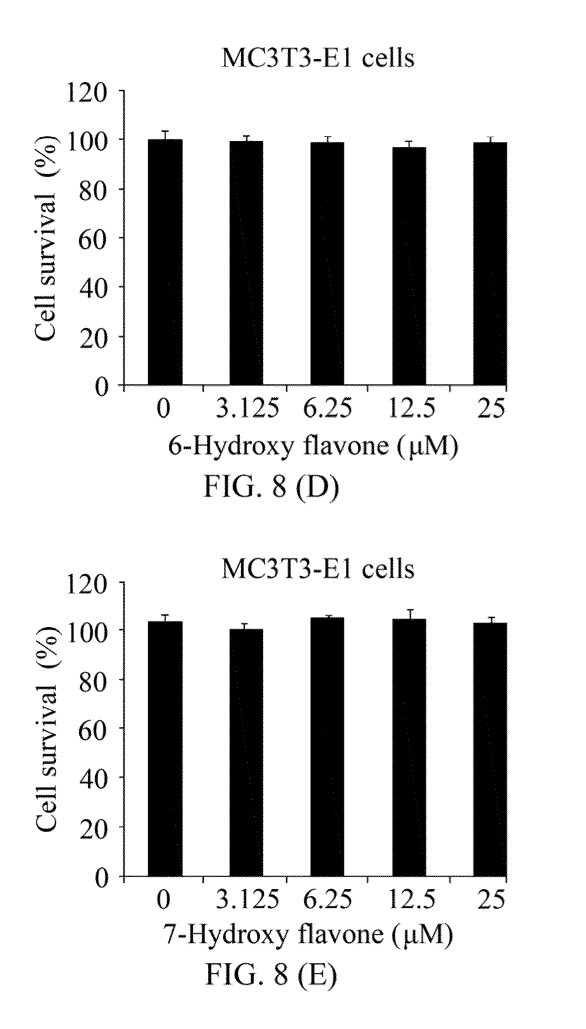

In an embodiment, different concentrations of flavone, 6-methoxy flavone, 7-methoxy flavone, 6-hydroxy flavone and 7-hydroxy flavone are respectively added into 3,000 MC3T3-E1 cells per well in the cultured medium for 1 day. Next, MTT reagent is used to detect the OD value of cells so as to know the growth situation of MC3T3-E1 cells. The result shows that all of them do not affect the growth of MC3T3-E1 cells, as shown in FIGS. 8(A)-8(E). That is, all of them have no toxicity to MC3T3-E1 cells.

Effects of Methoxy Flavones of the Present Invention on Calcium Deposition of MC3T3-E1 Cells

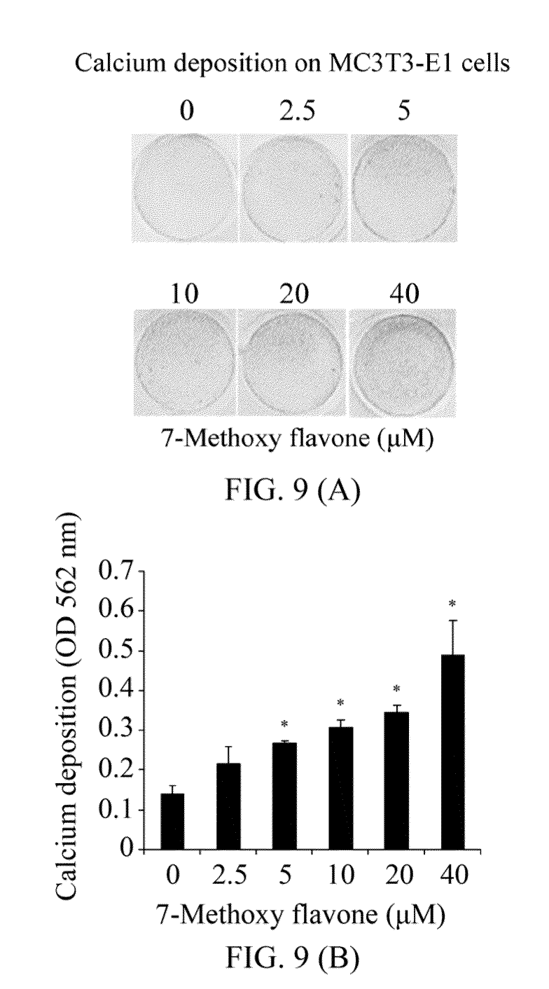

In an embodiment, different concentrations (2.5 μM, 5 μM, 10 μM, 20 μM and 40 μM) of 7-methoxy flavone are respectively added into the cultured medium of MC3T3-E1 cell incubated for 20 days, wherein the cultured medium with 7-methoxy flavone is changed every 4 days. Next, Alizarin Red S stain is used to observe the calcification situation of MC3T3-E1 cells, and OD value at 562 nm is measured for quantitative analysis. FIGS. 9(A) and 9(B) show that 7-methoxy flavone can effectively enhance calcium deposition of MC3T3-E1 cells at the concentration at 5 μM, 10 μM, 20 μM and 40 μM.

Effects of Methoxy Flavones and Hydroxyl Flavones of the Present Invention on Differentiation of SaOS-2 Cells

In an embodiment, flavone, 7-methoxy flavone, 6-hydroxy flavone, and 7-hydroxy flavone with 10 μM are respectively added into SaOS-2 cells (human osteosarcoma cells) in DMEM medium (10% FBS, 1× antibiotic-antimycotic solution) for 7 day, then performing the calcium staining for calcium deposition (CaP assay). The results show that 7-methoxy flavone, 6-hydroxy flavone, and 7-hydroxy flavone significantly enhance calcium deposition of SaOS-2 cells (data not shown).

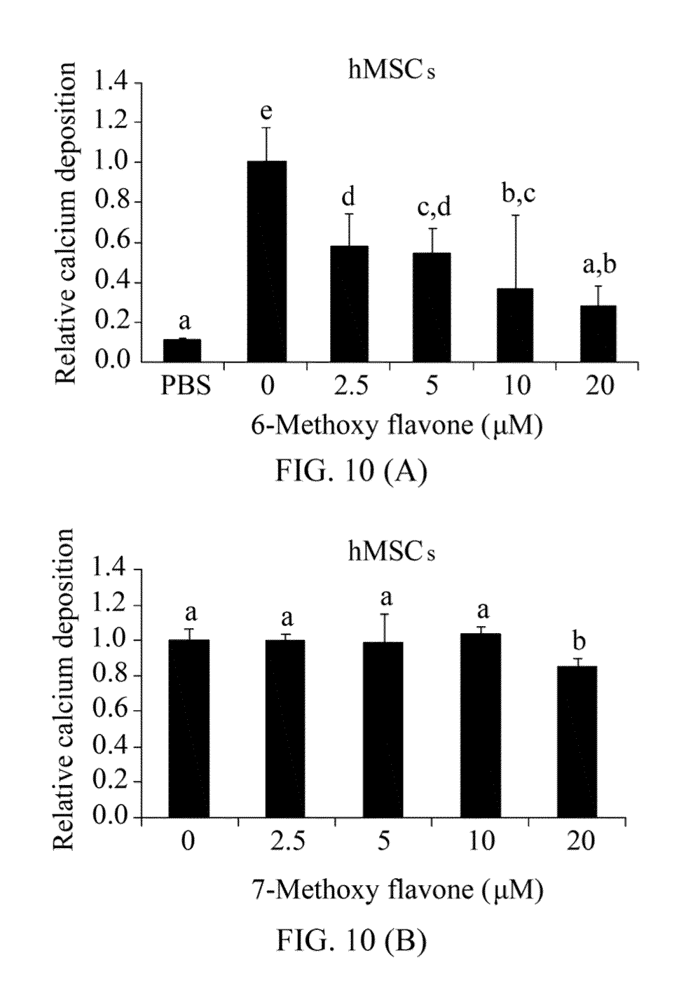

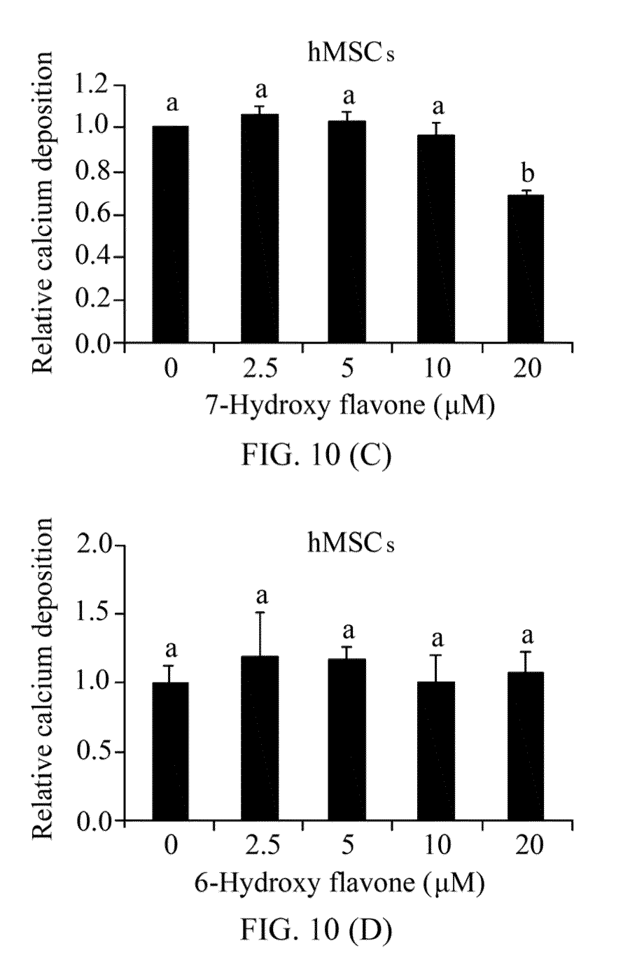

Moreover, the present invention also measures the effects of methoxy flavones and hydroxyl flavones on human mesenchymal stem cells (hMSCs). As shown in FIGS. 10(A)-10(D), 6-methoxy flavone greatly inhibits calcium deposition of hMSCs; 7-methoxy flavone and 7-hydroxy flavone inhibit calcium deposition on hMSCs only at high concentration; and 6-hydroxy flavone still does not inhibit calcium deposition even at high concentration.

In summary, it can be found that the compound comprising methoxy flavones and hydroxyl flavones of the present invention can enhance osteoblast differentiation and inhibiting osteoclast differentiation, and should have the potential to become parts of beneficial remedy for preventing from or therapeutic treatment for osteoporosis.

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope of all such changes and modifications as are within the true spirit and scope of the exemplary embodiments of the present invention.

What is claimed is:

1. A method of preventing and treating osteoporosis, comprising: enhancing osteoblast differentiation and inhibiting osteoclast differentiation by administering a pharmaceutically effective amount of a composition comprising 6-hydroxy flavone compound or 7-methoxy flavone compound, or at least one pharmaceutically acceptable salt thereof to a subject.

2. The method as claimed in claim 1, wherein the pharmaceutically effective concentration of 7-methoxy flavone is 3-60 μM.

3. The method as claimed in claim 2, wherein the pharmaceutically effective concentration of 7-methoxy flavone is further 5-50 μM.

4. The method as claimed in claim 1, wherein the pharmaceutically effective concentration of 6-hydroxy flavone is 3-60 μM.

5. The method as claimed in claim 4, wherein the pharmaceutically effective concentration of 6-hydroxy flavone is further 5-50 μM.
